(19) United States
(12) Patent Application Publication
(10) Pub. No.: US 2010/0217124 A1
(43) Pub. Date: Aug. 26, 2010

(54) ULTRASOUND IMAGING SYSTEM AND METHOD USING MULTILINE ACQUISITION WITH HIGH FRAME RATE

(75) Inventor: Clifford Cooley, Seattle, WA (US)

Correspondence Address: PHILIPS INTELLECTUAL PROPERTY & STANDARDS, P.O. BOX 3001, Briarcliff Manor, NY, 10510-8001, US

(73) Assignee: KONINKLIJKE PHILIPS ELECTRONICS, N.V., EINDHOVEN (NL)

(21) Appl. No.: 12/304,285

(22) PCT Filed: Jun. 20, 2007
(86) PCT No.: PCT/IB2007/052384

§ 371 Date: Dec. 11, 2008

Related U.S. Application Data
(60) Provisional application No. 60/805,922, filed on Jun. 27, 2006.

(51) Int. Cl. A61B 8/14

(52) U.S. Cl. 600/443

(57) ABSTRACT

An ultrasound imaging system includes an ultrasound probe having an array of transducer elements (12) divided into a plurality of contiguous transmit sub-apertures (14 a,b,c,d,e). A plurality of transmitters coupled to the sub-apertures of the ultrasound transducer apply respective transmit signals to the sub-apertures at different frequencies and with delays that cause respective transmit beams emanating from the sub-apertures to overlap each other in a region of interest (20). A multiline beamformer coupled to the transducer elements processes signals corresponding to ultrasound echoes to output image signals. A processor receives the image signals from the multiline beamformer and outputs image data corresponding to the image signals. The image data are processed by an image processor to output corresponding display signals that are applied to a display.

This invention relates to ultrasound imaging systems, and, more particularly, to an ultrasound imaging system that acquires images using multiline acquisition techniques.

Ultrasonic diagnostic imaging systems produce images of the interior of the body by transmitting ultrasonic waves which are steered and focused along transmit beams. Echoes are received from along the transmit beam path, and are used to produce an image of the structure or motion encountered along the beam path. A number of adjacently transmitted beams and their echoes will interrogate a planar region of the body, and the echoes can be used to produce a planar image of the body. The beams may also be transmitted adjacent to each other in three dimensions through a volumetric region, and the resulting echoes used to produce a three dimensional image of structures or flow in the volumetric region.

Ultrasound images are traditionally obtained by generating a transmit beam and then receiving echoes from the area or volume isonified by the transmit beam. An adjacent area or volume is then isonified by a transmit beam and echoes are again received from the isonified area or volume. In this manner, the area or volume from which echoes are received is sequentially scanned. Unfortunately, the rate at which echoes can be received is limited by the time required for the transmit beam to propagate and the resulting echoes to return from tissues in the area or volume being examined. As a result, the “frame rate,” i.e., the rate at which an entire image can be acquired, is limited. Limited frame rate can be a problem, particularly when imaging moving tissues. The problem of limited frame rate is even more severe for three dimensional ultrasound imaging in which transmit beams must be scanned in two dimensions.

One approach to increasing the frame rate of ultrasound images has been to use “multiline” beamformers to acquire ultrasound echoes. In multiline beamforming, a relatively wide transmit beampattern is used to isonify an area or volume, and the resulting echoes are simultaneously received along several spaced-apart receive lines. Multiline beamforming can provide high frame rates without reducing line density because multiple lines of echoes can be simultaneously received for each transmit beam. As a result, it is possible to obtain real-time images of moving tissues, even in three dimensions, in many cases.

As mentioned above, multiline imaging requires a transmit beampattern that is wide enough to encompass several receive lines. A large transmit beampattern is conventionally generated by using a transmit aperture that is much smaller than the receive apertures used to form the multiple receive lines. The conventional means for providing these transmit beampatterns is to use a number of transducer elements to form the transmit beam that is smaller than the number of transducer elements used to form each receive line. Unfortunately, since the power of the transmit beam is generally proportional to the combined area of the transducer elements generating the transmit beam, it is difficult to generate a transmit beam with good tissue penetration from a small aperture. As a result of the limited power of transmit beams used in conventional multiline ultrasound imaging systems, signals corresponding to the echoes received along each line may have a low signal-to-noise ratio, thereby sometimes resulting in poor image quality. This problem is even more serious in three dimensional multiline imaging systems because the transmit aperture must be small in two dimensions for the transmit beampattern to be wide in two dimensions.

There is therefore a need for a multiline ultrasound imaging system that can generate large, high power transmit beampatterns, thereby providing high quality ultrasound images at high frame rates.

An ultrasound imaging system and method are described which include an ultrasound probe that directs at least two transmit beams from respective sub-apertures into an area of interest. At least some of the transmit beams overlap each other in the area of interest. All of the overlapping transmit beams contain ultrasound at different frequencies. Ultrasound echoes from multiple lines in the area of interest are then received and processed by a multiline beamformer. The received ultrasound echoes are then processed to generate image data. The image data are then used to display an ultrasound image.

In the drawings:

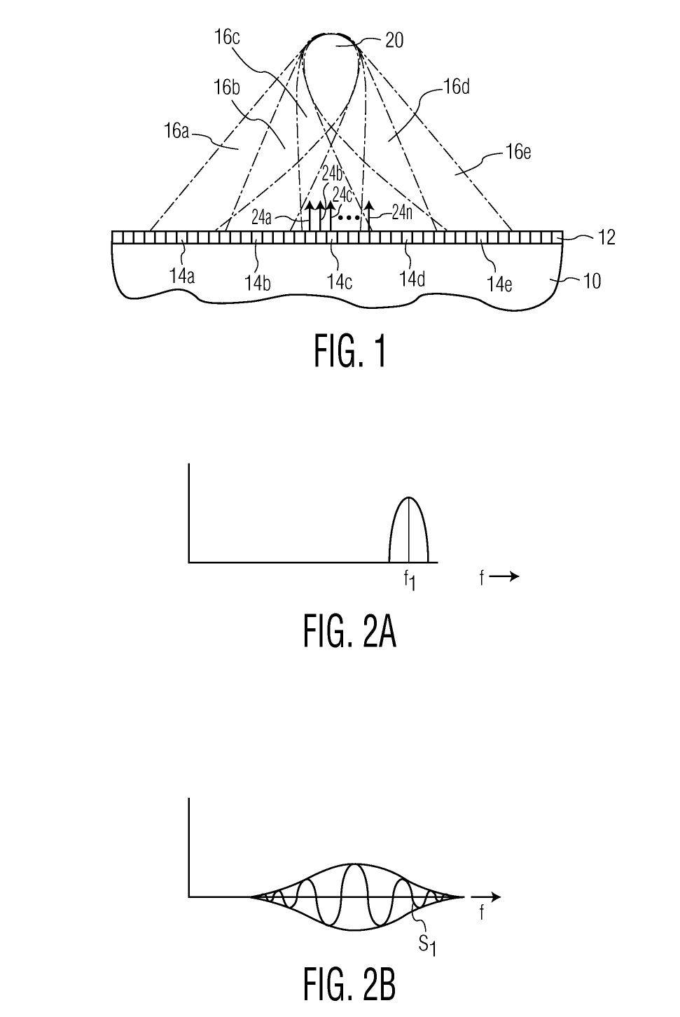

FIG. 1 is a schematic view illustrating one example of a technique for generating wide, high power transmit beam for multiline imaging.

FIGS. 2A and 2B illustrate a pulse and its sub-band in accordance with the principles of the present invention.

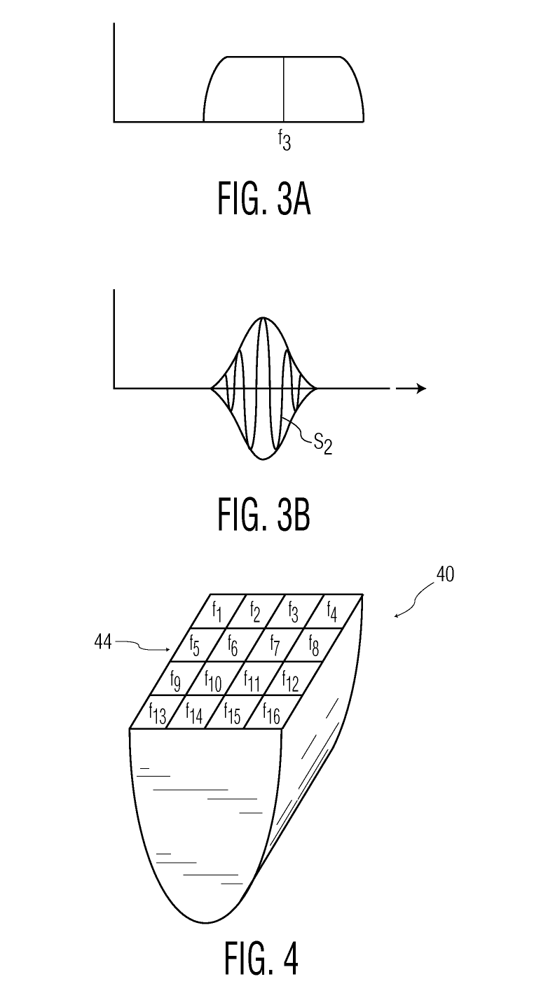

FIGS. 3A and 3B illustrate the result of combining sub-bands in accordance with the principles of the present invention.

FIG. 4 is an isometric view of a two dimensional ultrasound transducer that can be used to generate a three dimensional ultrasound image using a multiline beamforming techniques according to one example of the invention.

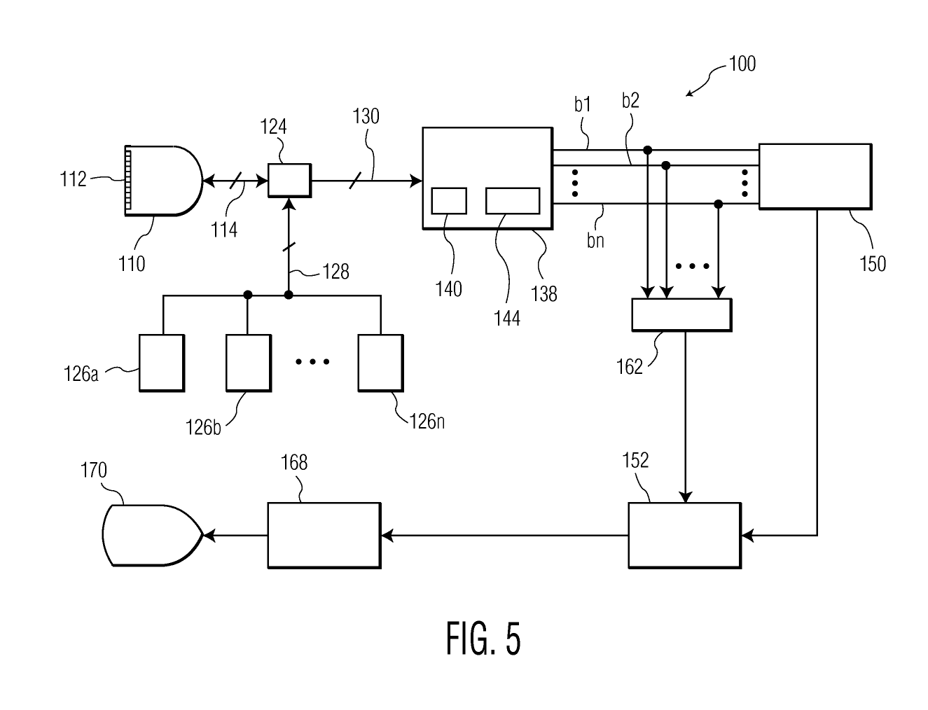

FIG. 5 is a block diagram of an ultrasound imaging system according to one example of the invention.

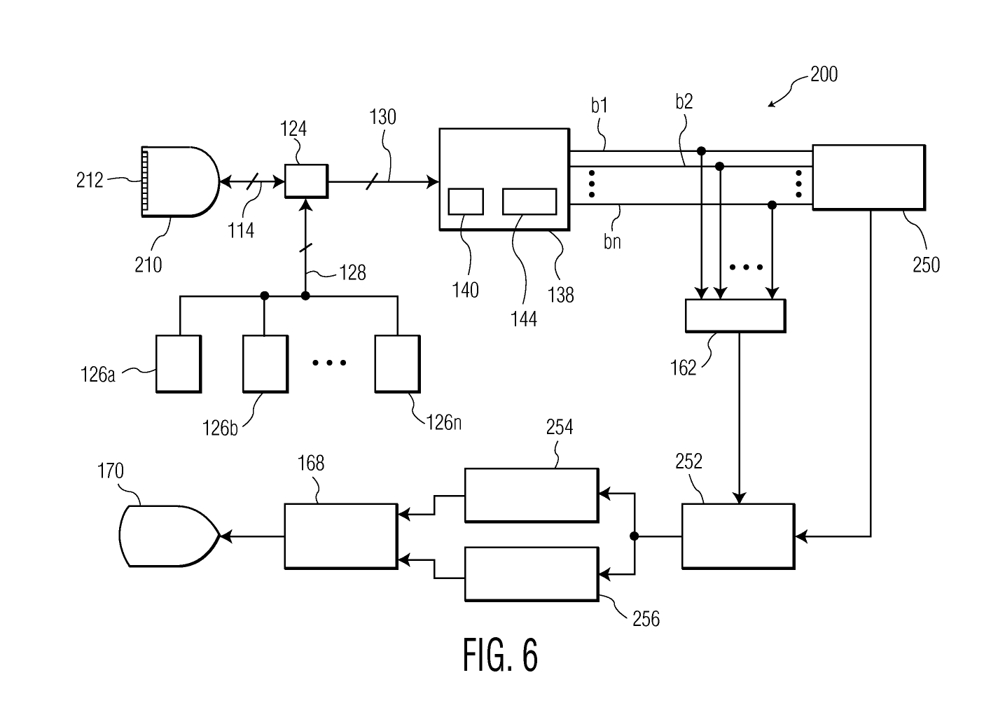

FIG. 6 is a block diagram of an ultrasound imaging system according to another example of the invention.

One example of a technique for generating large, high power transmit beams to allow multiline beamforming is shown in FIG. 1. An ultrasound probe 10 includes transducer elements 12 divided into five sub-apertures 14a,b,c,d,e. The transducer elements 12 forming the first sub-aperture 14a use respective transmit signals at a first frequency f₁ to generate a first transmit beampattern 16a. The transmit signals in the first sub-aperture 14a have respective delays that cause the beampattern 16a to be steered to the right. The transducer elements forming the second sub-aperture 14b use respective transmit signals at a second frequency f₂ and at respective delays to generate a second transmit beampattern 16b that is steered to the right to a lesser extent than the first transmit beampattern 16a is steered to the right. The transducer elements 12 forming the third sub-aperture 14c use respective transmit signals at a third frequency f₃ and with respective delays to generate a third transmit beampattern 16c that is perpendicular to the transducer elements 12. Similarly, the transducer elements forming the fourth and fifth apertures 14d,e, respectively, transmit respective beampatterns 16d,e at respective fourth and fifth frequencies f₄ and f₅ and at respective steering directions which steer the beampatterns to the left to differing degrees. As a result, the transmit beampatterns 16a,b,c,d,e are all focused in an area 20 of interest. In the limit, each element can be a sub-aperture, with frequencies continually changing as one progresses across the array of elements. It is from this area 20 that echoes are received to form multiple receive lines 24a-n.

The use of multiple transmit beams at different frequencies has several advantages. First, by using different frequencies for the transmit beams 16a,b,c,d,e, the signals in the beams do not constructively and destructively interfere with each other to provide unintended beamforming effects. Second, the amplitude of the ultrasound in the area 20 of interest is the sum of the individual amplitudes of all of the sub-aperture transmit beams. In the example shown in FIG. 1, the peak amplitude of the ultrasound in the area 20 is approximately five times the peak amplitude of a single sub-aperture transmit beam. In accordance with the principles of the present invention, this peak amplitude is achieved over a laterally wide transmit beamwidth suitable for multiline reception. Third, as explained in greater detail below, each transmit pulse has an effective pulse length that is longer than the length of a pulse typically used to produce the conventional multiline “fat beam.” A longer transmit pulse causes each resulting sub-band to be narrower in bandwidth as compared to the typical multiline fat beam. The sum of a plurality of such overlapping sub-bands spans a desired broad bandwidth with echo amplitudes also summed, resulting in good resolution and signal-to-noise ratio. Although the example shown in FIG. 1 uses an ultrasound probe that generates five transmit beams, a probe generating at least two overlapping transmit beampatterns will also provide these advantages, albeit to a greater or lesser degree.

The manner in which the overlapping transmit beampatterns of different frequencies in the area of interest 20 provides a broad effective bandwidth will now be explained with reference to FIGS. 2A and 2B, and FIGS. 3A and 3B. As shown in FIG. 2A, a pulse of ultrasound in a single transmit beampattern has a frequency spectrum in a narrow range centered at fᵢ. FIG. 2B shows a time domain signal S₁ corresponding to the frequency spectrum shown in FIG. 2A. It should be noted that, due to the relatively long duration of the signal S₁, the bandwidth of the signal S₁ is relatively narrow about the f₁ center frequency.

In contrast to the signal S₁ shown in FIGS. 2A,B, the combination of the five transmit beampatterns 16a,b,c,d,e shown in FIG. 1 have a frequency spectrum centered at f₃ that is quite wide, as shown in FIG. 3A. The corresponding effective signal S₂ of the combined sub-bands, shown in FIG. 3B, has a relatively short pulse-length. In order to provide a fairly constant, continuous frequency spectrum, the frequencies of the ultrasound used in the overlapping transmit beampatterns should be contiguous without any spectral gaps. Also, the frequencies should preferably increase in a linear manner from one side of an ultrasound probe to the other, although such is not required. By using apertures in which the frequency increases or decreases in a linear manner from one side to the other, the only effect of receiving echoes from the transmit beams emanating from different apertures is a slight difference in the focus depth from each transmit beam. The combined waveform is substantially the same throughout the whole main lobe in the lateral direction, but the effective depth is shifted axially by a small amount in time/depth in the lateral direction across the beam. This effect is not significant in relation to axial resolution and needs no correction to make a good image, although it can be taken into account for depth registration during coherent processing of the signals. If the frequencies are not distributed in a linear manner from side to side in the combined waveform, then although the combined bandwidth will be substantially the same throughout the combined transmit beam main lobe, the combined waveform shape and length in time will depend on lateral position within the transmit beam main lobe. By using a suitable filter on receive which depends on lateral position within the transmit beam, all of the receive multiline waveforms can be compressed to substantially the same short waveform. An example of a suitable filter is a matched filter matched to the signal expected from a point target at every image point.

An example of a two dimensional ultrasound transducer 40 that can be used to generate a three dimensional ultrasound image using a multiline beamformer is shown in FIG. 4. The transducer 40 has a transducer face 44 divided into 16 segments, each of which transmits ultrasound at a respective frequency f₁₋₁₆ with a relatively wide transmit beampattern. Transmit beampatterns overlap to insonify a volume of interest beneath the transducer face 44. Echoes are then received from multiple receive lines in the volume of interest.

An ultrasound imaging system 100 according to one example of the invention is shown in FIG. 5. The system 100 includes an ultrasonic probe 110 capable of two dimensional imaging using a one dimensional or line array of transducer elements 112. The transducer elements 112 are coupled through respective lines 114 to a transmit/receive switch 124 that is operated by a conventional control circuit (not shown). The transducer elements 112 are arranged in transmit sub-arrays, and each sub-array is connected by the switch 124 to a respective transmitter 126a-n through respective lines 128. The transmitters 126a-n each generate transmit signals at a respective frequency, and the signals that each transmitter 126a-n applies to the transducer elements 112 in its respective sub-array are appropriately timed to steer the transmit beampatterns as explained above with reference to FIG. 1. The transmit beampatterns therefore overlap in a two dimensional area of interest beneath the transducer elements 112.

After overlapping transmit beampatterns have been generated by the probe 110, the switch 124 connects the transducer elements 112 through respective signal lines 130 to a multiline beamformer 138 of conventional design. Echo signals received by the transducer elements 112 in response to the transmit beams are then coupled to the multiline beamformer 138. The beamformer 138 processes the received echo signals to provide echo data for multiple receive lines. A suitable multiline beamformer for this purpose is described in U.S. Pat. No. 6,695,783. The multiline beamformer 138 may also include matched filters 140 to correct for the slight defocusing in time of echo signals received from the overlapping transmit beams, as explained above. Additionally, the multiline beamformer 138 may include a depth dependent matched filter 144 to obtain an extended depth of field and thereby achieve optimum depth resolution, as also explained above. The echo data corresponding to the multiple receive lines formed by the multiline beamformer 138 are output from the beamformer 138 on separate beamformer output lines b1, b2, . . . bn, but may be output in other formats, such time-interleaved signals on fewer lines, frequency multiplexed on a single line, or output as an optical signal through an optical fiber.

The echo data corresponding to the multiple receive lines can be applied to a Doppler processor 150, which processes the echo data into two dimensional Doppler power or velocity information. The two dimensional Doppler information is stored in a 2D data memory 152, from which it can be displayed in various formats. The echo data for the multiple receive lines can be coupled to a B-mode detector 162, where the echo signals are envelope detected. Data corresponding to the detected echo data can then be stored in the 2D data memory 152.

The two dimensional image data stored in the 2D data memory 152 may be processed for display by several conventional means. Signals corresponding to the resulting images are coupled to an image processor 168, from which they are displayed on an image display 170.

In another example of the invention, an ultrasound imaging system 200 shown in FIG. 6 is capable of generating ultrasound images showing anatomical structures in a three dimensional volumetric region. The imaging system 200 includes many of the same components that are used in the two dimensional imaging system 100 shown in FIG. 5. Therefore, in the interest of brevity, an explanation of the structure and function of the components that operate in essentially the same manner will not be repeated. The system 200 differs from the system 100 by using an ultrasound probe 210 having a two dimensional array of transducer elements 212. As a result, the transmit beampatterns overlap in a three dimensional area of interest.

The system 200 also differs from the system 100 by using a three dimensional Doppler processor 250 rather than a two dimensional Doppler processor 150, which generates three dimensional Doppler information. Additionally, the system 200 uses a 3D data memory 252 to store the three dimensional Doppler information, from which it can be displayed in various formats such as a 3D power Doppler display. For example, the three dimensional image data stored in the 3D data memory 252 may be processed for display by producing multiple 2D planes of the volume. Such planar images of a volumetric region are produced by a multi-planar reformatter 254. The three dimensional image data may also be rendered to form a 3D display by a volume renderer 256. The resulting images are coupled to the image processor 168, from which they are displayed on the image display 170.

Although the present invention has been described with reference to the disclosed embodiments, persons skilled in the art will recognize that changes may be made in form and detail without departing from the spirit and scope of the invention. For instance the inventive broad beam effect can be formed on receive by transmitting broad bandwidth signals into the image field, receiving different sub-bands of frequencies, then combining the received sub-band signals. Such modifications are well within the skill of those ordinarily skilled in the art. Accordingly, the invention is not limited except as by the appended claims.

We claim:

1. A method of acquiring an ultrasound image, comprising:
directing at least two transmit beams into an area of interest, at least some of the transmit beams overlapping each other in the area of interest, the overlapping transmit beams being at different bands of frequencies;
receiving ultrasound echoes from multiple lines in the area of interest;
processing the received ultrasound echoes to generate image data; and
using the image data to display the ultrasound image.

2. The method of claim 1 wherein the frequency spectra of all of the overlapping transmit beams are contiguous without any significant spectral gaps.

3. The method of claim 1 wherein the frequency of each overlapping beam increases in a linear manner from one side of the area of interest to another.

4. The method of claim 1 wherein the act of directing at least two transmit beams into an area of interest comprises directing at least two transmit beams into a two dimensional area of interest.

5. The method of claim 1 wherein the act of directing at least two transmit beams into an area of interest comprises directing at least two transmit beams into a three dimensional area of interest to allow a volumetric ultrasound image to be displayed.

6. A method of multiline beamforming, comprising:
directing at least two transmit beams into an area of interest, at least some of the transmit beams overlapping each other in the area of interest, the overlapping transmit beams being at different bands of frequencies;
receiving ultrasound from multiple regions in the area of interest; and
processing signals corresponding to the ultrasound echoes received from each of the regions to form respective lines of receive signals.

7. The method of claim 6 wherein the frequency spectra of all of the overlapping transmit beams are contiguous without any significant spectral gaps.

8. The method of claim 6 wherein the frequency of each overlapping beam increases in a linear manner from one side of the area of interest to another.

9. The method of claim 6 wherein the act of directing at least two transmit beams into an area of interest comprises directing at least two transmit beams into a two dimensional area of interest.

10. The method of claim 6 wherein the act of directing at least two transmit beams into an area of interest comprises directing at least two transmit beams into a three dimensional area of interest to allow a volumetric ultrasound image to be displayed.

11. An ultrasound imaging system, comprising:
an ultrasound probe having an array of transducer elements, the transducer elements being divided into a plurality of transmit sub-apertures;
a plurality of transmitters coupled to the transmit sub-apertures, the transmitters applying to the respective transmit sub-apertures a transmit signal at a respective frequency band that is different from the frequency bands of the transmit signals that the other transmitters apply to the other respective transmit sub-apertures, the signals that each of the transmitters apply to the respective transmit sub-apertures being focused so that respective transmit beams emanating from the transmit sub-apertures overlap each other in a region of interest;
a multiline beamformer coupled to the transducer elements, the multiline beamformer processing signals corresponding to ultrasound echoes to output image signals corresponding to respective receive lines in the region of interest;
a signal processor coupled to receive the image signals from the multiline beamformer, the signal processor outputting image data corresponding to the image signals;
an image processor coupled to receive the image data from the signal processor, the image processor generating display signals corresponding to the image data; and
a display coupled to receive the display signals from the image processor, the display being operable to use the display signals to provide an ultrasound image corresponding to the display signals.

12. The ultrasound imaging system of claim 11 wherein the multiline beamformer comprises a matched filter.

13. The ultrasound imaging system of claim 12 wherein the matched filter comprises a depth dependent matched filter.

14. The ultrasound imaging system of claim 11 wherein the array of transducer elements in the ultrasound probe comprise a one dimensional array of transducer elements.

15. The ultrasound imaging system of claim 11 wherein the array of transducer elements in the ultrasound probe comprise a two dimensional array of transducer elements.

16. The ultrasound imaging system of claim 11 wherein the signal processor comprises a Doppler processor.

17. The ultrasound imaging system of claim 11 wherein the signal processor comprises a B-mode detector.

18. The ultrasound imaging system of claim 11 wherein the frequencies of the transmit signals that the respective transmitters apply to the respective sub-apertures are contiguous from one transmit sub-aperture to the next from one side of the array to the other.

19. The ultrasound imaging system of claim 11 wherein the frequencies of the transmit signals that the respective transmitters apply to the respective transmit sub-apertures increase in a linear manner from one transmit sub-aperture to the next from one side of the array to the other.
